(19) United States
(12) Patent Application Publication
(10) Pub. No.: US 2017/0000463 A1
(43) Pub. Date: Jan. 5, 2017

(54) ULTRASONIC DIAGNOSTIC APPARATUS

(71) Applicant: HITACH, LTD., Tokyo (JP)

(72) Inventors: Nobuhiko FUJII, Tokyo (JP); Tatsuya HAYASHI, Tokyo (JP)

(73) Assignee: Hitachi, Ltd., Tokyo (JP)

(21) Appl. No.: 15/125,559

(22) PCT Filed: Oct. 8, 2014
(86) PCT No.: PCT/JP2014/076941

§ 371 Date: Sep. 12, 2016

(30) Foreign Application Priority Data
Mar. 13, 2014 (JP) 2014-050412

(51) Int. Cl. A61B 8/08; G01S 7/52

(52) U.S. Cl. CPC A61B 8/5269; A61B 8/5207; A61B 8/085; G01S 7/52049

(57) ABSTRACT

A plurality of frames are generated by performing a pre-scan while sequentially setting a plurality of receive delay data based on a plurality of in vivo sound velocities. An optimum sound velocity calculation unit performs waveform analysis for each luminance waveform along a beam scanning direction on each of the frames. An optimum sound velocity map is obtained by mutually comparing a plurality of results of the waveform analysis of the plurality of frames. A control unit calculates the receive delay data for the main scan on the basis of the optimum sound velocity map. Specifically, waveform analysis for a high luminance portion (peak portion) and waveform analysis for a low luminance portion (recess portion) are performed in the waveform analysis. Thus, an optimum sound velocity map for the high luminance portion and an optimum sound velocity map for the low luminance portion are obtained.

### TECHNICAL FIELD

The present invention relates to an ultrasonic diagnostic apparatus, and in particular to techniques to determine the optimum in vivo sound velocity that defines a delay process condition.

### BACKGROUND

Ultrasonic diagnostic apparatuses are used in medical fields for forming ultrasonic images by transmitting and receiving ultrasonic waves to and from living bodies. Ultrasonic waves are typically transmitted and received by two or more oscillators. Specifically, in transmitting ultrasonic waves, transmission signals which accord with a transmission delay process condition corresponding to a transmission focal point are supplied to the oscillators to form transmission beams. In receiving ultrasonic waves, reflection waves (echoes) from inside a living body are received by the oscillators. A phasing addition process according to a reception delay process condition is applied to the received signals, which are output from the oscillators, to generate reception beam data. After the phasing addition process, an ultrasonic image is formed based on the reception beam data. It should be noted that, during reception, a reception dynamic focus is typically applied in which the reception focal point is dynamically changed from a proximity point to a deeper point along the beam axis.

The phasing addition process during reception is described in detail below. To apply a delay process to reception signals, delay data (delay time) defining a delay process condition are used. The delay data are used to achieve a reception dynamic focus and reception beam scan. The delay data are formed from data sets corresponding to the respective oscillators. To calculate the delay data, a fixed value is typically used as the in vivo sound velocity; for example, 1530 m/s.

### CITATION LIST

### Patent Literature

Patent Document 1: JP 2008-264531 A

### SUMMARY

### Technical Problem

However, the in vivo velocity of ultrasonic waves varies depending on the properties of the in vivo tissues. Use of the delay data calculated on the assumption of a fixed sound velocity may fail to achieve an appropriate reception focus, depending on the actual diagnostic status, reducing reception sensibility, and image resolution. In this regard, Patent Document 1 discloses an ultrasonic diagnostic apparatus which obtains variations of contrast values while changing the sound velocity used for calculation of the delay data separately for respective small areas on the scanned surface, and defines the sound velocity with the highest contrast for each of the small areas as the optimum sound velocity for that small area. The contrast values indicate difference in luminance. Accordingly, this method is sufficient for calculating the optimum sound velocity for tissues having a high luminance, such as calcified tissues. However, low-luminance tissues, such as infiltrating cancer, have a low luminance by nature (low echo tissues having a certain expansion). Accordingly, methods using the contrast values are inappropriate for calculating the optimum sound velocity for low-luminance tissues. Such a method may set a sound velocity that is improper for observing low-luminance tissues. As described here, it has been impossible to generate delay process conditions which are appropriate for observing tissues with different properties (for example, high-luminance tissues and low-luminance tissues) and enhancing images of these tissues together. Although reception processes are described above, the same issue exists also for transmission processes.

An object of the present invention is to determine the optimum in vivo sound velocity which can be used to obtain a delay process condition in an ultrasonic diagnostic apparatus. Another object of the present invention is to generate a delay process condition suitable for observing tissues with different properties.

### Solution to Problem

An ultrasonic diagnostic apparatus according to the present invention includes a generator which generates two or more frames by repeatedly scanning a subject with an ultrasonic beam; a pre-scan controller which sequentially sets, to the generator, two or more delay process conditions in trial based on two or more tentative sound velocities such that two or more tentative frames are generated; a waveform analyzer which performs a waveform analysis for at least one reference data sequence along a preset direction in each of the tentative frames to evaluate a sharpness of an image, and thereby obtains two or more waveform analysis results for the two or more tentative frames; an optimum sound velocity calculator which calculates an optimum sound velocity based on the two or more waveform analysis results; and a main scan controller which sets, to the generator, a delay process condition for a main scan based on the optimum sound velocity.

According to the above configuration, two or more frames with different tentative sound velocities are generated by sequentially applying, in trial, two or more delay process conditions which have been calculated based on the two or more tentative sound velocities. The sharpness of an image changes depending on the in vivo sound velocity which defines the delay process conditions. Accordingly, the sharpness of an image can be evaluated by applying a waveform analysis to the two or more frames with different tentative sound velocities. This evaluation by the waveform analysis corresponds to the evaluation of the two or more in vivo sound velocities. Thus, among two or more in vivo sound velocities, the optimum in vivo sound velocity which can sharpen the image can be determined by using the waveform analysis result.

It is preferable that the preset direction is a beam scanning direction; and that the waveform analyzer performs a local waveform analysis at two or more positions in the reference data sequence to obtain a local waveform analyzed value sequence which forms the waveform analysis result.

It is preferable that the waveform analyzer performs a waveform analysis separately for each of the reference data sequences arranged in a depth direction in each of the tentative frames to obtain a local waveform analyzed value matrix which forms the waveform analysis result.

It is preferable that the waveform analyzer includes a first waveform analyzer which performs a first waveform analysis on the two or more reference data sequences in each of the tentative frames to obtain two or more first local waveform analyzed value matrices corresponding to the two or more tentative frames; and a second waveform analyzer which performs a second waveform analysis on the two or more reference data sequences in each of the tentative frames to obtain two or more second local waveform analyzed value matrices corresponding to the tentative frames, the second waveform analysis being different from the first waveform analysis, wherein the optimum sound velocity calculator calculates the optimum sound velocity based on the two or more first local waveform analyzed value matrices and the two or more second local waveform analyzed value matrices.

It is preferable that in the first waveform analysis, sharpness is analyzed for each convex peak portion; and in the second waveform analysis, sharpness is analyzed for each concave low-luminance portion.

It is preferable that in the second waveform analysis, gradients of respective edges of the low-luminance portion are separately analyzed and sharpness of the entire low-luminance portion is analyzed based on the gradients.

The peak portion corresponds to, for example, a high-luminance tissue in a living body (for example, a calcified tissue). The present invention evaluates the sharpness of an image of a high-luminance tissue by recognizing a peak portion as a single entity. The optimum in vivo sound velocity to sharpen an image of a high-luminance tissue can be determined using the evaluation result. A low-luminance portion corresponds to a low-luminance tissue in a living body (for example, an infiltrating cancer). A low-luminance tissue includes a portion with a rapid change in luminance (boundary portion of the low-luminance portion) and a portion with a gradual change in luminance. The luminance gradients reflect the sharpness of the image. Accordingly, a portion with a rapid change in luminance is more suitable for evaluation of the sharpness of an image than a portion with a gradual change in luminance. Therefore, for a low-luminance portion, a portion with a rapid change (boundary portion of the low-luminance portion) is more preferably evaluated. Regarding a high-luminance tissue and a low-luminance tissue having different properties, such an evaluation of sharpness by a method suitable for each property can determine the in vivo sound velocity suitable for each tissue.

It is preferable that the optimum sound velocity calculator includes a function to generate a first optimum sound velocity map indicating an optimum sound velocity at each position on a beam scanning surface based on the two or more first local waveform analyzed value matrices; and a function to generate a second optimum sound velocity map indicating an optimum sound velocity at each position on the beam scanning surface based on the two or more second local waveform analyzed value matrices, wherein the optimum sound velocity for the main scan is obtained based on the first optimum sound velocity map and the second optimum sound velocity map.

It is preferable that the optimum sound velocity calculator includes a function to generate a composite map by synthesizing the first optimum sound velocity map and the second optimum sound velocity map. The synthesizing process (integration process) includes, for example, averaging of the sound velocities, application of the median of the sound velocities, and application of the maximum value of the sound velocities.

It is preferable that the optimum sound velocity calculator includes a function to calculate one or more optimum sound velocities that define the delay process condition for the main scan by applying an aggregation process to the two or more optimum sound velocities constituting the composite map.

It is preferable that the waveform analyzer includes a first low-pass filter which applies a first filtering process to the two or more reference data sequences in each of the tentative frames; and a second low-pass filter which applies a second filtering process to the two or more reference data sequences in each of the tentative frames, the second filtering process having a stronger effect than the first filtering process, wherein the first waveform analyzer applies a first waveform analysis to the two or more reference data sequences in each of the tentative frames after the first filtering process; and the second waveform analyzer applies a second waveform analysis to the two or more reference data sequences in each of the tentative frames after the second filtering process. This can remove noises and prevent the luminance gradients of peak portions from being gradual, reducing or preventing the decrease in the accuracy of evaluation of the sharpnesses of the peak portions. In addition, regarding low luminance portions, noises can be effectively removed.

### Advantageous Effects of Invention

The present invention enables determination of the optimum sound velocity to be used for calculation of a delay process condition in an ultrasound diagnostic apparatus.

### BRIEF DESCRIPTION OF DRAWINGS

FIG. 1 is a block diagram showing an example of an ultrasonic diagnostic apparatus according to an embodiment of the present invention;

FIG. 2 is a schematic diagram showing an example of a high-luminance tissue and a low-luminance tissue;

FIG. 3A is a schematic diagram showing an example of a high-luminance tissue and a low-luminance tissue;

FIG. 3B is a graph showing an example of changes in luminance in a high-luminance tissue;

FIG. 3C is a graph showing an example of changes in luminance in a low-luminance tissue;

FIG. 4A is a diagram showing a relationship between a reception focal point and changes in luminance in a high-luminance tissue;

FIG. 4B is another diagram showing a relationship between a reception focal point and changes in luminance in a high-luminance tissue;

FIG. 4C is still another diagram showing a relationship between a reception focal point and changes in luminance in a high-luminance tissue;

FIG. 5A is a graph used to describe changes in luminance in a low-luminance tissue;

FIG. 5B is another graph used to describe changes in luminance in a low-luminance tissue;

FIG. 5C is still another graph used to describe changes in luminance in a low-luminance tissue;

FIG. 6 is a schematic diagram showing an example of a reception frame sequence;

FIG. 7A is a diagram used to describe how to obtain sharpness of a high-luminance tissue;

FIG. 7B is another diagram used to describe how to obtain sharpness of a high-luminance tissue;

FIG. 8 is a diagram used to describe how to obtain high-luminance portion sound velocity mapping data;

FIG. 9 is a schematic diagram showing an example of the high-luminance portion sound velocity mapping data;

FIG. 10A is a diagram used to describe how to obtain sharpness of a low-luminance portion;

FIG. 10B is another diagram used to describe how to obtain sharpness of a low-luminance portion;

FIG. 11 is a schematic diagram showing an example of low-luminance portion sound velocity mapping data;

FIG. 12 is a diagram used to describe an integration process of sound velocity mapping data;

FIG. 13 is a flowchart showing a main routine of an ultrasonic diagnostic apparatus according to an embodiment of the present invention;

FIG. 14 is a flowchart showing an optimum sound velocity determination process;

FIG. 15 is a flowchart showing an optimum sound velocity determination process according to Variation 1; and

FIG. 16 is a flowchart showing an optimum sound velocity determination process according to Variation 2.

### DESCRIPTION OF EMBODIMENTS

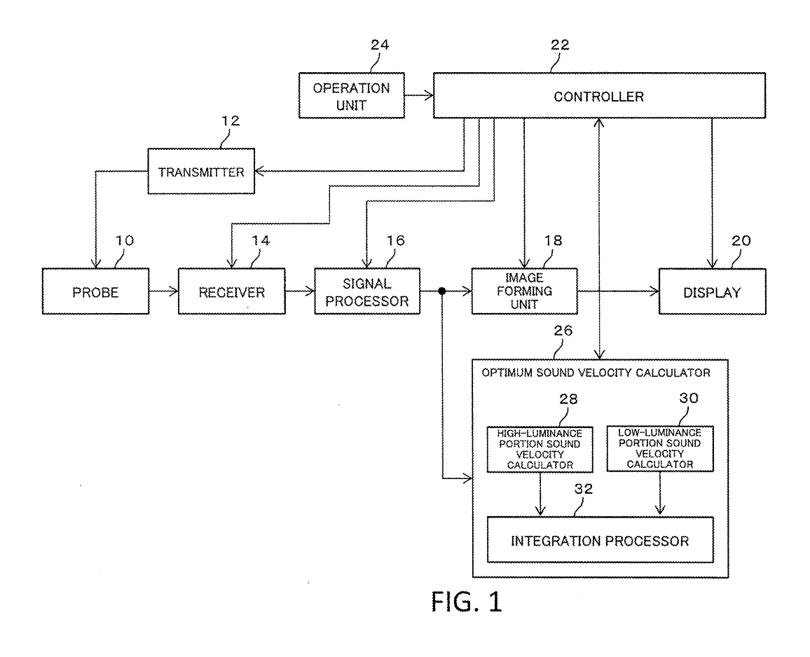

FIG. 1 is an example of an ultrasonic diagnostic apparatus according to an embodiment of the present invention. Ultrasonic diagnostic apparatuses are installed at medical facilities such as a hospital and are used to form ultrasonic images by transmitting and receiving ultrasonic waves to and from human bodies.

In FIG. 1, a probe 10 is a wave transceiver which transmits and receives ultrasonic waves to and from a diagnostic area. The probe 10 includes two or more oscillators which transmit and receive ultrasonic waves. The oscillators form ultrasonic beams. The ultrasonic beams are repeatedly and electronically scanned to progressively form a beam scanning surface. As an electronic scanning system, electronic sector scanning and electronic linear scanning are known. As the probe 10, a one-dimensional probe in which oscillators are aligned in a single line in a predetermined direction or a two-dimensional probe in which oscillators are arranged in two dimensions is used. As the probe 10, an oscillator having a semiconductor called “capacitive micromachined ultrasonic transducer (cMUT)” (IEEE Trans. Ultrason., Ferroelect. Freq. Contr., Vol 45, pp. 678-690, May 1998) may also be used.

A transmitter 12 is a transmission beam former. During transmission, the transmitter 12 forms and transmits transmission signals to each oscillator of the probe 10 by applying a delay process corresponding to each of the oscillators and supplies the transmission signals to each oscillator. This forms a transmission beam of ultrasonic waves. During transmission, transmission beam focus control is performed. In addition, the transmitter 12 is provided with a bore control function. During transmission, when reflection waves from a living body are received by the probe 10, the probe 10 outputs reception signals to a receiver 14.

The receiver 14 is a reception beam former. During reception, the receiver 14 forms reception beams by applying a phasing addition process or the like to the reception signals obtained from the oscillators. Specifically, the receiver 14 forms reception beams by applying a delay process in accordance with a delay process condition set for each oscillator to the reception signals from each of the oscillators, and further applies an addition process to the reception signals obtained from the oscillators. The delay process condition is defined by reception delay data (delay time). During reception, reception dynamic focus control is performed. A controller 22 supplies a reception delay data set (delay time set) corresponding to the oscillators. The controller 22 calculates the delay time based on the in vivo sound velocities.

The transmitter 12 and the receiver 14 perform electronic scanning using transmission beams and reception beams (ultrasonic beams). This forms a beam scanning surface. The beam scanning surface corresponds to beam data which form reception frames (reception frame data). Each piece of the beam data is formed by echo data aligned in a depth direction. By repeating electron scanning using the ultrasonic beams, reception frames aligned in a time axis are output from the receiver 14. These reception frames form a reception frame sequence.

A transmission/reception switch (not shown) is provided for switching between a transmission function and a reception function. During transmission, the transmission/reception switch supplies transmission signals from the transmitter 12 to each oscillator. During reception, the transmission/reception switch supplies receptions signals from the oscillators to the receiver 14.

A signal processor 16 is a module for processing the reception frame sequences. The signal processor 16 may include, for example, a detector circuit, a signal compression circuit, a gain adjustment circuit, and a filter process circuit. The signal compression circuit compresses a reception signal of a dynamic range as large as, for example, the twentieth power of two, to a relatively small range. The signal compression may be based on a logarithmic function, an exponential function, or a sigmoid function. The filter process circuit performs an enhancement process, for example, to sharpen boundaries.

An image forming unit 18 includes a digital scan converter which provides a coordinate conversion function, an interpolation process function, and other functions. The image forming unit 18 forms a display frame sequence including two or more display frames based on the reception frame sequence. The individual display frame in the display frame sequence shows B-mode tomographic image data. For example, with a convex type probe 10, the image forming unit 18 converts rectangular data to a fan-shaped ultrasonic image. The display frame sequence is output to and displayed on a display 20 such as a liquid crystal monitor. In this way, the B-mode tomographic image can be displayed in real time as a video image. The image forming unit 18 may include a gamma correction processor, which corrects display tone using a gamma curve. The display 20 may use analog or digital output display techniques, so long as the display 20 can display ultrasonic images which can be used for diagnosis by an operator.

The controller 22 controls operations of each element shown in FIG. 1. An ultrasonic diagnostic apparatus according to the present embodiment has a test operation mode to determine an optimum in vivo sound velocity in addition to a normal main scan mode. The controller 22 has a control function in the test operation mode. Specific control operations are described further below.

An operation unit 24 is connected to the controller 22. The operation unit 24 may include a keyboard, a trackball, or the like. A user can input parameters for capturing ultrasonic images through the operation unit 24. According to the present embodiment, a user can also provide instructions to perform the test operation mode through the operation unit 24. The test operation mode can be instructed by a user before or during a normal ultrasonic diagnostics operation. The controller 22 is an example of a “pre-scan controller” and a “main-scan controller.”

An optimum sound velocity calculator 26 operates at the time of pre-scan before the main scan to determine the optimum sound velocity which is used as a basis for the delay data calculation (delay process condition calculation) for the main scan. Specifically, the optimum sound velocity calculator 26 includes a high-luminance portion sound velocity calculator 28, a low-luminance portion sound velocity calculator 30, and an integration processor 32. The optimum sound velocity calculator 26 operates when determining the optimum sound velocity; in other words, in the test operation mode. In the test operation mode, the optimum sound velocity calculator 26 receives a reception frame sequence generated by applying two or more pieces of reception delay data which have been calculated based on two or more in vivo sound velocities. The optimum sound velocity calculator 26 determines the optimum sound velocity for calculating the reception delay data based on the reception frame sequence. The optimum sound velocity calculator 26 is an example of a “waveform analyzer” and an “optimum sound velocity calculator.” The high-luminance portion sound velocity calculator 28 is an example of a “first waveform analyzer” and the low-luminance portion sound velocity calculator 30 is an example of a “second waveform analyzer.” Each element of the optimum sound velocity calculator 26 is described below.

The high-luminance portion sound velocity calculator 28 determines, based on the reception frame sequence, the optimum sound velocity to sharpen an image of a high luminance tissue such as a calcified tissue. For each reception frame sequence, the high-luminance portion sound velocity calculator 28 obtains inflection points in a luminance waveform (a waveform showing a change in a luminance (echo strength) in the scan direction of the ultrasonic beam) and calculates a luminance gradient between neighboring inflection points. Next, the high-luminance portion sound velocity calculator 28 calculates sharpness at a peak portion by integrally evaluating luminance gradients at both sides of a peak portion of the luminance waveform (a convex portion of the luminance waveform) for each reception frame. Then, the high-luminance portion sound velocity calculator 28 determines the optimum sound velocity for sharpening an image of high luminance tissue based on the sharpness of each reception frame. The high-luminance portion sound velocity calculator 28 determines, for each coordinate (pixel), the reception frame having the highest sharpness in the reception frame sequence and determines the in vivo sound velocity corresponding to the reception frame as the optimum sound velocity for high luminance tissues. The high-luminance portion sound velocity calculator 28 may also set, as an invalid value, the in vivo sound velocity of a coordinate having a luminance gradient equal to or less than a threshold. The high-luminance portion sound velocity calculator 28 generates high-luminance portion sound velocity mapping data indicating the optimum sound velocity for each coordinate.

The low-luminance portion sound velocity calculator 30 determines, based on the reception frame sequence, the optimum sound velocity to sharpen an image of a low-luminance tissue (low echo tissue expanded to some extent) such as an infiltrating cancer. Regarding each of the reception frame sequence, the low-luminance portion sound velocity calculator 30 obtains inflection points in a luminance waveform (a waveform showing a change in a luminance in the scan direction of the ultrasonic beam) and calculates a luminance gradient between neighboring inflection points. The low-luminance portion sound velocity calculator 30 evaluates a luminance gradient of each edge (a portion with a rapid change in luminance) at both sides of a low-luminance portion (a concave portion) of a luminance waveform to individually calculate the sharpness at each of the edges. The edge in a low-luminance portion corresponds to a boundary of the low-luminance portion. Then, the low-luminance portion sound velocity calculator 30 determines the optimum sound velocity for sharpening an image of the low-luminance tissue based on the sharpness of each reception frame. The low-luminance portion sound velocity calculator 30 determines, for each coordinate, the reception frame having the highest sharpness in the reception frame sequence and determines the in vivo sound velocity corresponding to the reception frame as the optimum sound velocity for low-luminance tissues. The low-luminance portion sound velocity calculator 30 may also set, as an invalid value, the in vivo sound velocity having a luminance gradient equal to or less than a threshold. Then, the low-luminance portion sound velocity calculator 30 generates low-luminance portion sound velocity mapping data indicating the optimum sound velocity for each coordinate.

The integration processor 32 generates integrated sound velocity mapping data by integrating the high-luminance portion sound velocity mapping data and the low-luminance portion sound velocity mapping data. This integrated sound velocity mapping data are supplied to the controller 22 for calculation of the reception delay data.

The controller 22 has a function to calculate a reception delay data set based on the optimum sound velocity. In the present embodiment, the controller 22 calculates the reception delay data for each reception point depth based on the integrated sound velocity mapping data in order to achieve a reception dynamic focus for each orientation of the beam. The reception delay data define delay time differences among reception signals to converge reception beams at a reception point. In the present embodiment, a reception delay data set is calculated based on the optimum sound velocity. As another example, two or more reception delay data sets corresponding to in vivo sound velocities may be defined in advance. In this case, when the optimum sound velocity is determined, the controller 22 selects a reception delay data set which corresponds to the determined optimum sound velocity. A transmission delay data set may also be calculated.

The elements shown in FIG. 1 except for the probe 10 may be realized using hardware resources such as processors and electronic circuits. In order to realize the elements, a device such as a memory may be used, as required. Further, the elements other than the probe 10 may be realized by computers. Specifically, all or part of the elements except for the probe 10 may be realized by interaction between hardware resources of a computer (such as a CPU, memory, and hardware) and software (a program) defining operations of CPU or the like. The program may be stored in a storage device (not shown) via a storage medium such as a CD and DVD, or through a communication path such as a network. As another example, the elements except for the probe 10 may be realized by digital signal processors (DSPs) and field programmable gate arrays (FPGAs).

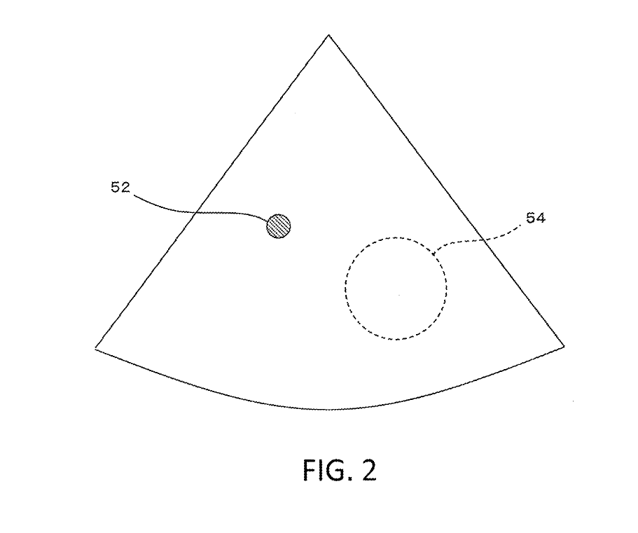

Next, specific processes performed by the optimum sound velocity calculator 26 according to the present embodiment are described. First, by referring to FIG. 2, images of tissues shown in a B-mode tomographic image are described. The B-mode tomographic image shown in FIG. 2 shows, as examples, a high-luminance tissue 52 such as a calcified tissue and a low-luminance tissue 54 (a low echo tissue having a certain expansion) such as an infiltrating cancer. The high-luminance tissue 52 and the low-luminance tissue 54 have properties different from each other.

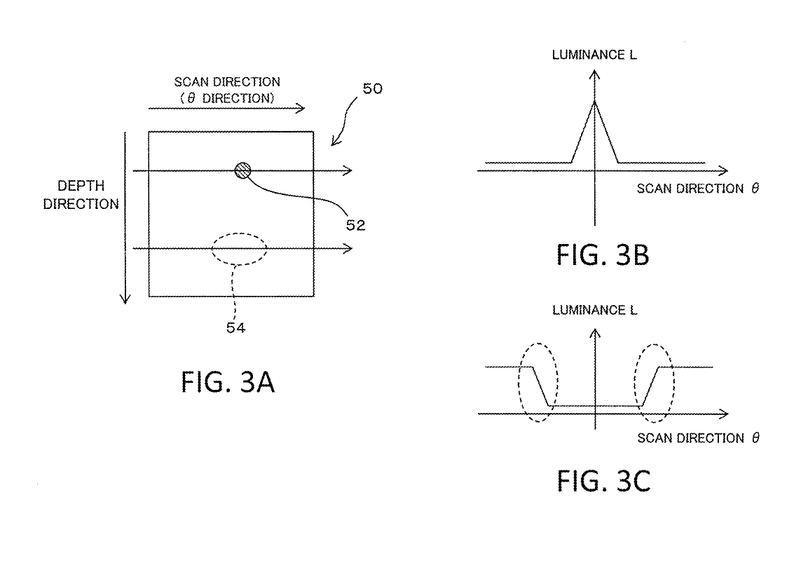

By referring to FIGS. 3A, 3B, and 3C, changes in luminance of a high-luminance tissue and a low-luminance tissue are described below. A reception frame 50 shown in FIG. 3A shows the high-luminance tissue 52 and the low-luminance tissue 54. One direction in the reception frame 50 represents the scan direction θ of an ultrasonic beam and the other direction represents the depth direction. The luminance waveform shown in FIG. 3B represents changes in luminance of the high-luminance tissue 52 in the scan direction θ. In the luminance waveform, the peak portion (convex portion) represents the high-luminance tissue 52. The luminance waveform shown in FIG. 3C represents changes in luminance of the low-luminance tissue 54 in the scan direction θ. In the luminance waveform, the low-luminance portion having a low luminance L with almost no change (concave portion) represents the low-luminance tissue 54. As encircled in the broken lines in FIG. 3C, changes in the luminance L are large at boundaries (edges) of the low-luminance portion. As described above, in a luminance waveform, the high-luminance tissue 52 forms a peak portion and the low-luminance tissue 54 forms a concave portion. The high-luminance tissue 52 and the low-luminance tissue 54 have luminance change properties that differ from each other.

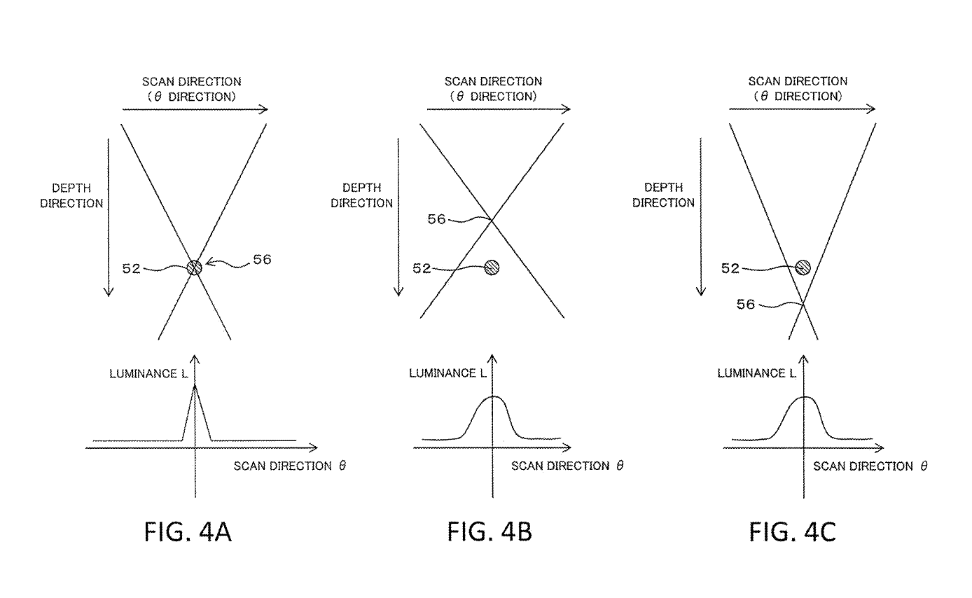

A relationship between a focal point of an ultrasonic beam and the luminance L of a tissue is now described. FIGS. 4A, 4B, and 4C show a relationship between a reception focal point and the luminance L of a high-luminance tissue. As shown in FIG. 4A, when the in vivo sound velocity for calculating reception delay data is equal to the actual sound propagation velocity in a living body, the target position (the position of the high-luminance tissue 52) and a reception focal point 56 can be equal to each other. In this case, the peak portion representing the high-luminance tissue 52 in the luminance waveform in the scan direction becomes very steep; in other words, the gradient of the luminance L becomes large. Accordingly, the space resolution of the image in the scan direction θ can be improved. In contrast, when the in vivo sound velocity for calculating the reception delay data is slower or faster than the actual sound propagation velocity in a living body, the reception focal point 56 is positioned shallower or deeper than the target position (the position of the high-luminance tissue 52), as shown in FIGS. 4B and 4C. In this case, the peak portion in the luminance waveform becomes less steep and the space resolution of an image in the scan direction θ decreases. As a result, the image of the high-luminance tissue 52 becomes dull.

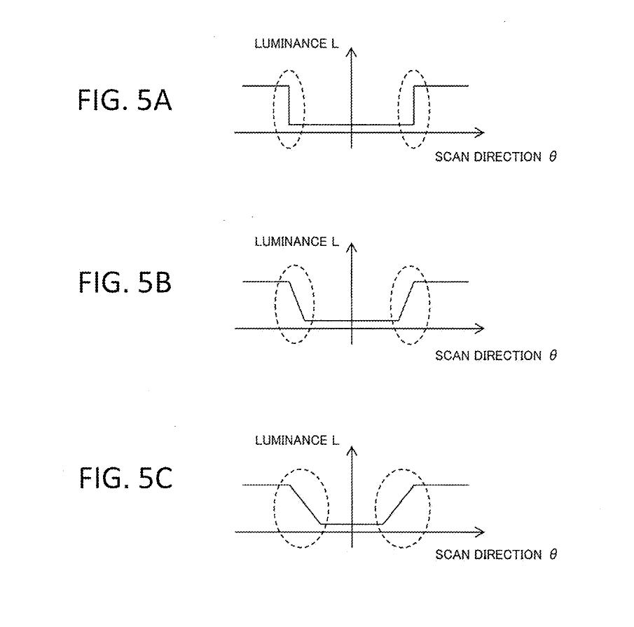

FIGS. 5A, 5B, and 5C show relationships between a reception focal point and the luminance L of a low-luminance tissue. As shown in FIG. 5A, when the in vivo sound velocity for calculating the reception delay data is equal to the actual sound propagation velocity in a living body, the edges (encircled in the broken lines in FIG. 5A) of the concave portion representing the low-luminance tissue 54 in the luminance waveform in the scanning direction θ become steep; in other words, the gradient of the luminance L becomes large. Accordingly, the space resolution of the image in the scan direction θ can be improved. In contrast, when the in vivo sound velocity for calculating the reception delay data is slower or faster than the actual sound propagation velocity in a living body, the gradation of the edges in the luminance waveform becomes less steep and the space resolution of an image in the scan direction θ decreases, as shown in FIGS. 5B and 5C. As a result, the image of the low-luminance tissue 54 becomes dull.

As shown in FIGS. 4A to 4C and 5A to 5C, the resolution of an image in the scan direction θ changes in accordance with the in vivo sound velocity for calculating the reception delay data. The present embodiment focuses on this point. The in vivo sound velocities optimum for a high-luminance tissue and a low-luminance tissue are separately determined by analyzing the changes in luminance (luminance gradient) in the scan direction θ.

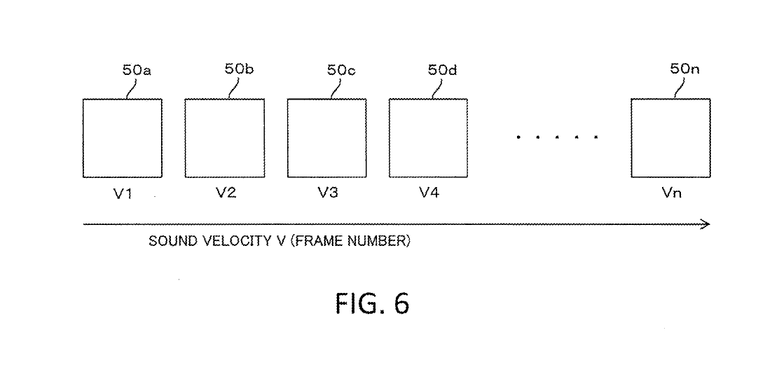

FIG. 6 shows an example of a reception frame sequence which is generated in the operation test mode (pre-scan mode). The reception frames 50a, 50b, 50c, . . . , and 50n are data which are generated by sequentially applying two or more reception delay data sets calculated in accordance with an in vivo sound velocity V1, V2, V3, . . . , and Vn. The reception frames are generated from the same scanned surface; in other words, the same tissue structure. For example, the reception frame 50a is data which are generated by applying a reception delay data set calculated in accordance with the in vivo sound velocity V1. As described above, n reception frames having different in vivo sound velocities can be generated by setting n different in vivo sound velocities for calculation. In the test operation mode, the controller 22 sequentially supplies the reception delay data sets respectively corresponding to the in vivo sound velocities V1 to Vn to the receiver 14. The receiver 14 generates reception frames 50a to 50n by sequentially performing processes such as a phasing addition process on the reception signals in accordance with the reception delay data sets.

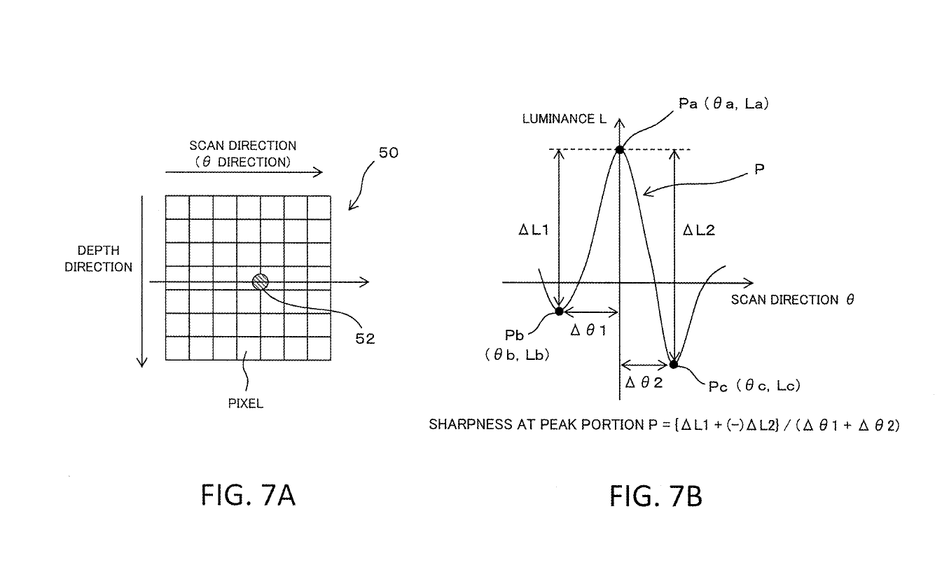

Next, by referring to FIGS. 7A and 7B, specific processes performed by the high-luminance portion sound velocity calculator 28 are described. The reception frame 50 shown in FIG. 7A includes the high-luminance tissue 52. The waveform shown in FIG. 7B is a part of a luminance waveform in the scan direction θ at the high-luminance tissue 52. Two or more luminance waveforms exist in the depth direction. The following processes are performed for each of these luminance waveforms. The high-luminance portion sound velocity calculator 28 detects inflection points of the luminance waveform, Pa (local maximum point), Pb (local minimum point), and Pc (local minimum point), and calculates luminance gradients (ΔL/Δθ) between neighboring inflection points. Then, the high-luminance portion sound velocity calculator 28 calculates the sharpness of the peak portion P in accordance with the luminance gradients on both sides of the top (local maximum point Pa) of the peak portion P (convex portion).

Specifically, the high-luminance portion sound velocity calculator 28 calculates the sharpness of the peak portion P by the following Equation (1):

Sharpness of Peak portion={ΔL1+(−)ΔL2}/(Δθ1+Δθ2) Equation (1)

ΔL1 is the difference (La−Lb) (>0) between the luminance La of the local maximum point Pa and the luminance Lb of the local minimum point Pb.

ΔL2 is the difference (Lc−La) (<0) between the luminance Lc of the local minimum point Pc and the luminance La of the local maximum point Pc.

Δθ1 is the difference between a position θa of the local maximum point Pa and a position θb of the local minimum point Pb in the scan direction θ. This difference represents the number of pixels between the position θa and the position θb.

Δθ2 is the difference between a position θa of the local maximum point Pa and a position θc of the local minimum point Pc in the scan direction θ. This difference represents the number of pixels between the position θa and the position θb.

It should be noted that the “pixel” corresponds to the coordinates (reception point or sample point) on the scanned surface. This also applies to the following descriptions.

(Δθ1+Δθ2) represents the width of the peak portion, and (ΔL1+(−)ΔL2) represents the luminance L of the peak portion. (ΔL1/Δθ1) represents the luminance gradient on one side of the peak portion P, and {(−)ΔL2/Δθ2} represents the luminance gradient on the other side of the peak portion P. Accordingly, the sharpness obtained by Equation (1) corresponds to an evaluation value when the peak portion P is recognized as a single convex portion. As described above, the high-luminance portion sound velocity calculator 28 obtains the sharpness of the peak portion P by recognizing the peak portion P formed between the bottom (local minimum point Pb) and the other bottom (local minimum point Pc) of the luminance waveform as the subject to be analyzed.

The high-luminance portion sound velocity calculator 28 applies the same sharpness for all the pixels (coordinates) in the peak portion P. In the example shown in FIG. 7B, the high-luminance portion sound velocity calculator 28 applies the sharpness obtained by Equation (1) as the sharpness for all the respective pixels between the local minimum point Pb and the local minimum point Pc. For example, when ten pixels exist between the local minimum point Pb and the local minimum point Pc, the high-luminance portion sound velocity calculator 28 applies the same sharpness for these ten pixels. Accordingly, all of the ten pixels have the same sharpness.

For each of the reception frames 50a to 50n shown in FIG. 6, the high-luminance portion sound velocity calculator 28 calculates the sharpness for each of the pixels.

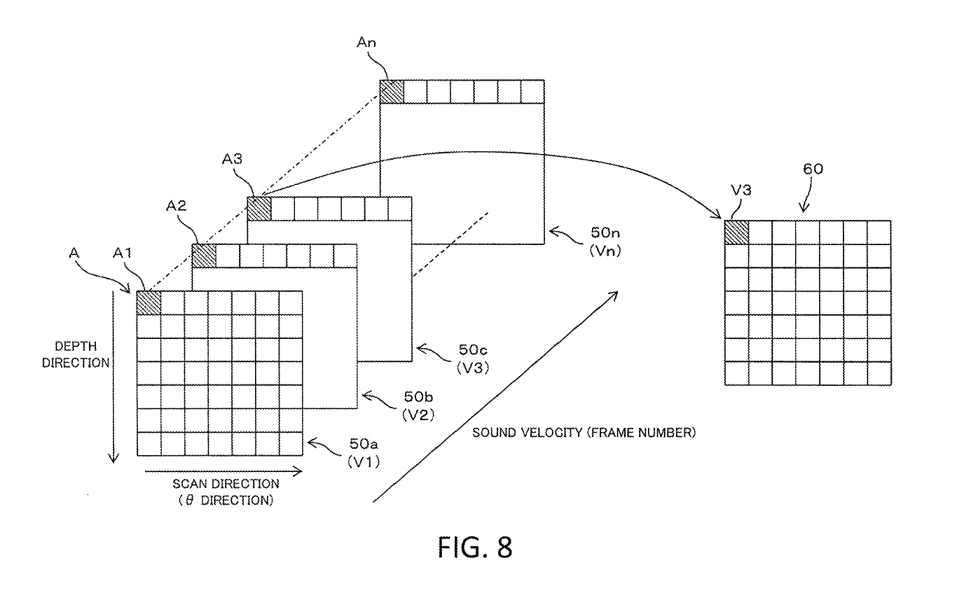

Then, the high-luminance portion sound velocity calculator 28 identifies, from the reception frames 50a to 50n, the reception frame which has the maximum sharpness, and determines the in vivo sound velocity corresponding to the identified reception frame as the optimum sound velocity for the high-luminance tissue. For example, as shown in FIG. 8, the high-luminance portion sound velocity calculator 28 compares the sharpness A1 to An of the same pixel A of the reception frames 50a to 50n. When, for example, the sharpness A3 of the reception frame 50c is the maximum among the sharpnesses A1 to An, the high-luminance portion sound velocity calculator 28 determines the in vivo sound velocity V3 of the frame 50c as the optimum sound velocity at pixel A. The high-luminance portion sound velocity calculator 28 determines the optimum sound velocity for each pixel and generates a high-luminance portion sound velocity mapping data 60 which shows the optimum sound velocity for each pixel.

As described above by referring to FIGS. 3A to 3C and 4A to 4C, the peak portion (convex portion) of a luminance waveform corresponds to a high luminance tissue. The sharpness of the peak portion changes in accordance with the in vivo sound velocity for calculating the reception delay data. Accordingly, identification of the reception frame with the maximum sharpness at the peak portion results in the determination of the optimum sound velocity which can sharpen the image of the high-luminance tissue.

The high-luminance portion sound velocity calculator 28 may invalidate the in vivo sound velocity of a pixel which has a sharpness of zero in any of the reception frames. Further, the high-luminance portion sound velocity calculator 28 may calculate the average of the sharpnesses of all the pixels in all the reception frames and invalidate the in vivo sound velocities of pixels which have the sharpnesses less than certain times lower than the average. This can remove noises, thereby suppressing reduction of accuracy in determination of the in vivo sound velocity.

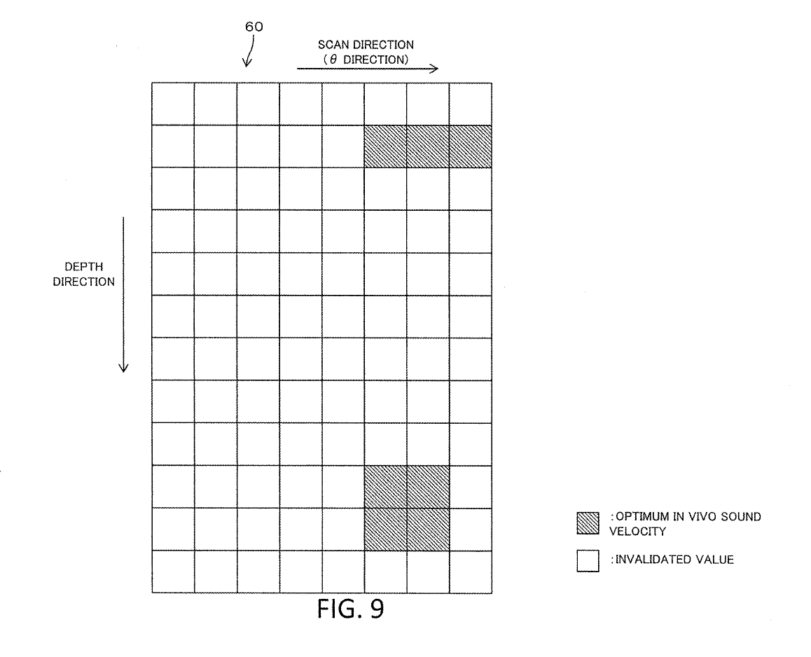

FIG. 9 shows an example of the high-luminance portion sound velocity mapping data 60. In the high-luminance portion sound velocity mapping data 60, the values of the pixels shown in hatching are optimum sound velocities determined by the high-luminance portion sound velocity calculator 28. The values of the pixels other than these hatched pixels are invalidated.

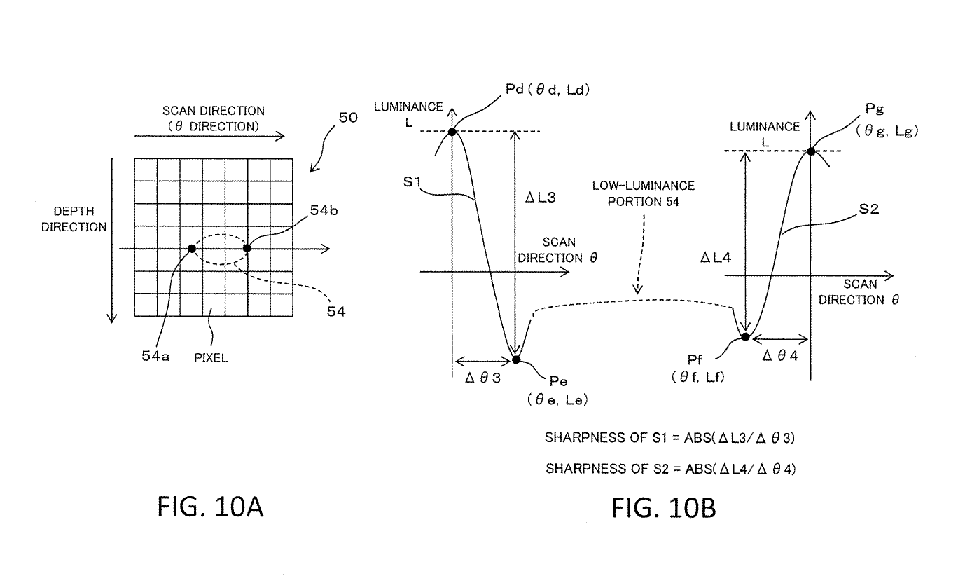

Next, by referring to FIGS. 10A and 10B, specific processes performed by the low-luminance portion sound velocity calculator 30 are described. The reception frame 50 shown in FIG. 10A includes a low-luminance tissue 54. The waveform shown in FIG. 10B is a part of a luminance waveform in the scan direction θ at the low-luminance tissue 54. The low-luminance portion sound velocity calculator 30 detects inflection points of the luminance waveform, Pd (local maximum point), Pe (local minimum point), Pf (local minimum point), and Pg (local maximum point), and calculates luminance gradients (ΔL/Δθ) between neighboring inflection points as the luminance gradients at the edges of the low-luminance portion (concaved portion). For example, the waveform between the local maximum point Pd and the local minimum point Pe corresponds to the edge S1 of the low-luminance portion, and the waveform between the local minimum point Pf and the local maximum point Pg corresponds to the other edge S2 of the low-luminance portion. The edge S1 corresponds to a boundary 54a of the low-luminance tissue 54, and the edge S2 corresponds to a boundary 54b of the low-luminance tissue 54. The low-luminance portion sound velocity calculator 30 calculates the luminance gradient separately for each of the edges S1, S2 on both sides of the low-luminance portion. Specifically, the low-luminance portion sound velocity calculator 30 calculates the luminance gradient of the edge S1 as the sharpness of the edge S1, and the luminance gradient of the edge S2 as the sharpness of the edge S2.

More specifically, with the gradient of the luminance waveform recognized in the scan direction θ, the low-luminance portion sound velocity calculator 30 calculates the absolute value of the luminance gradient of the falling portion (edge S1) of the luminance waveform, which is the absolute value of the luminance gradient (ΔL3/Δθ3) between the top (local maximum point Pd) and the bottom (local minimum point Pe) of the luminance waveform, as the sharpness of the edge S1. Similarly, the low-luminance portion sound velocity calculator 30 calculates the absolute value of the luminance gradient of the rising portion (edge S2) of the luminance waveform, which is the absolute value of the luminance gradient (ΔL4/Δθ4) between the bottom (local minimum point Pf) and the top (local maximum point Pg) of the luminance waveform, as the sharpness of the edge S2.

ΔL3 is the difference between the luminance Ld of the local maximum point Pd and the luminance Le of the local minimum point Pe (Le−Ld) (<0).

Δθ3 is the difference between a position θd of the local maximum point Pd and a position θe of the local minimum point Pe in the scan direction. Δθ3 represents the number of pixels between the position θd and the position θe.

ΔL4 is the difference between the luminance Lf of the local minimum point Pf and the luminance Lg of the local maximum point Pg (Lg−Lf) (>0).

Δθ4 is the difference between a position θf of the local minimum point Pf and a position θg of the local maximum point Pg in the scan direction. Δθ4 represents the number of pixels between the position θf and the position θg.

The low-luminance portion sound velocity calculator 30 applies the same sharpness for all the pixels for each edge. In the example shown in FIG. 10B, the low-luminance portion sound velocity calculator 30 applies the absolute value of the luminance gradient (ΔL3/Δθ3) as the sharpness of each pixel between the local maximum point Pd and the local minimum point Pe, and the absolute value of the luminance gradient (ΔL4/Δθ4) as the sharpness of each pixel between the local minimum point Pf and the local maximum point Pg. In other words, the sharpness of each pixel between the local maximum point Pd and the local minimum point Pe is the same value (ΔL3/Δθ3), and the sharpness of each pixel between the local minimum point Pf and the local maximum point Pg is also the same value (ΔL4/Δθ4).

The low-luminance portion sound velocity calculator 30 calculates the sharpness of each pixel for each of the reception frames 50a to 50n shown in FIG. 6.

Then, the low-luminance portion sound velocity calculator 30 identifies, from the reception frames 50a to 50n, the reception frame which has the maximum sharpness, and determines the in vivo sound velocity corresponding to the identified reception frame as the optimum sound velocity for low-luminance tissues. For example, when the luminance gradient of the reception frame 50a is the maximum for a certain pixel, the low-luminance portion sound velocity calculator 30 determines the in vivo sound velocity V1 of the reception frame 50a as the optimum sound velocity at the pixel. The low-luminance portion sound velocity calculator 30 determines the optimum sound velocity for each pixel and generates low-luminance portion sound velocity mapping data which shows the optimum sound velocity for each pixel.

As described above by referring to FIGS. 3A to 3C and 5A to 5C, the low-luminance portion (concave portion) of the luminance waveform corresponds to a low-luminance tissue. The sharpnesses of the edge portions change depending on the in vivo sound velocity for calculating the reception delay data. Accordingly, with the portion between the inflection points (between neighboring local minimum point and local maximum point) of the luminance waveform recognized as an edge portion, the identification of the reception frame with the maximum luminance gradient (sharpness) of the edge portion determines the optimum sound velocity which can sharpen an image of the low-luminance tissue.

The low-luminance portion sound velocity calculator 30 may invalidate the in vivo sound velocity of a pixel which has a luminance gradient (sharpness) of zero in any of the reception frames. Further, the low-luminance portion sound velocity calculator 30 may calculate the average of the luminance gradients of all the pixels in all the reception frames and invalidate the in vivo sound velocities of the pixels which have the luminance gradients less than certain times lower than the average. This can remove noises, suppressing reduction of accuracy in determination of the in vivo sound velocity.

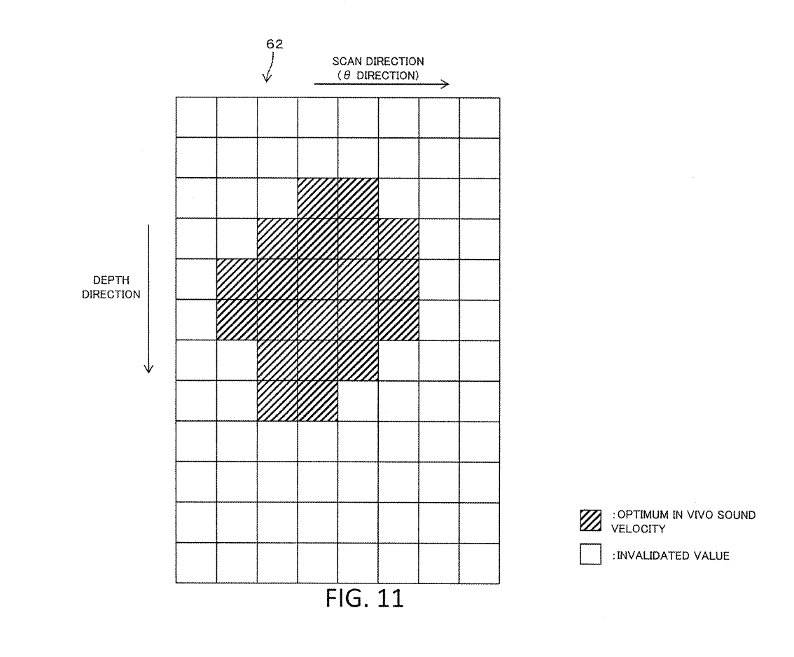

FIG. 11 shows an example of low-luminance portion sound velocity mapping data. In the low-luminance portion sound velocity mapping data 62, the values of pixels shown by hatching represent the optimum in vivo sound velocities identified by the low-luminance portion sound velocity calculator 30. The other pixel values are invalidated.

The high-luminance portion sound velocity calculator 28 and the low-luminance portion sound velocity calculator 30 may perform smoothing by applying a low-pass filter (LPF) to the reception frames such that the portions other than the subject portions (the peak portion, and edges in low-luminance portions) in the luminance waveform are not evaluated. The high-luminance portion sound velocity calculator 28 and the low-luminance portion sound velocity calculator 30 may determine the optimum sound velocity by calculating the luminance gradient (sharpness) for the reception frames after applying the low-pass filter. In this case, the high-luminance portion sound velocity calculator 28 applies, to the reception frames, a low-pass filter which is weaker than the low-pass filter for low-luminance tissues. In contrast, the low-luminance portion sound velocity calculator 30 applies, to the reception frames, a low-pass filter which is stronger than the low-pass filter for high-luminance tissues. For high-luminance tissues, sharpness at a peak portion is to be evaluated. Thus, if a stronger filter is applied, the gradient at a peak portion to be evaluated becomes less steep. This may reduce the accuracy of the evaluation of the sharpness. This is the reason why the high-luminance portion sound velocity calculator 28 applies a weaker low-pass filter. In contrast, an application of a stronger low-pass filter gives less effect to an expansion of a low-luminance portion, because a low-luminance portion is expanded to some degree. This is the reason why the low-luminance portion sound velocity calculator 30 applies the stronger low-pass filter to efficiently remove noises.

The high-luminance portion sound velocity calculator 28 and the low-luminance portion sound velocity calculator 30 calculate, for example, the sharpness of each pixel at each depth for a data sequence in the scan direction corresponding to each depth. Alternatively, the high-luminance portion sound velocity calculator 28 and the low-luminance portion sound velocity calculator 30 may calculate the sharpness of each pixel at a certain depth for a data sequence in the scan direction at a certain depth. Further, the high-luminance portion sound velocity calculator 28 and the low-luminance portion sound velocity calculator 30 may calculate the sharpness of each pixel within a region of interest (ROI) for a data sequence in the scan direction within the ROI. In this case, a reception delay data set based on a predetermined in vivo sound velocity may be applied to the regions other than the ROI.

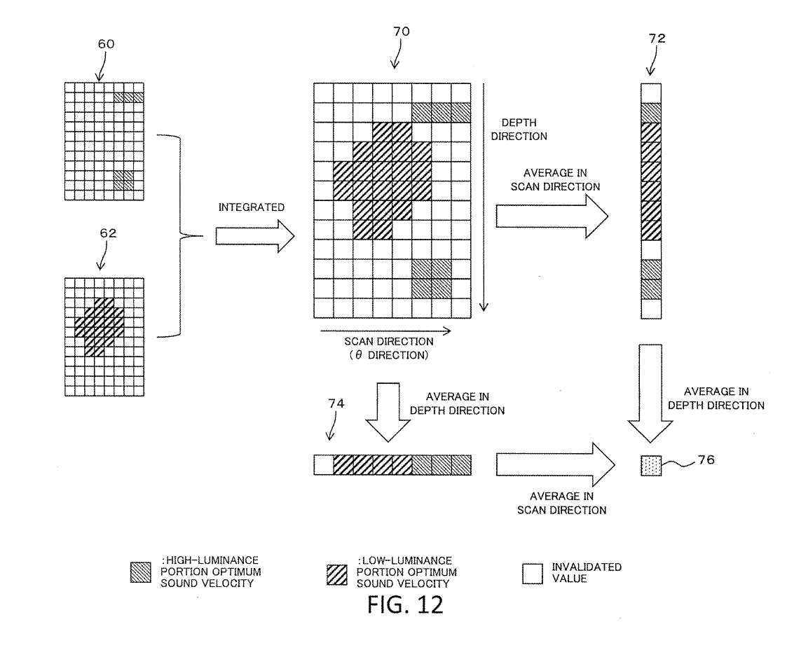

Next, by referring to FIG. 12, specific processes of the integration processor 32 are described. The integration processor 32 generates integrated sound velocity mapping data 70 by integrating the high-luminance portion sound velocity mapping data 60 and the low-luminance portion sound velocity mapping data 62. For example, the integration processor 32 generates the integrated sound velocity mapping data 70 by overwriting the high-luminance portion sound velocity mapping data 60 with the low-luminance portion sound velocity mapping data 62 to update the data. Alternatively, the integration processor 32 may generate the integrated sound velocity mapping data 70 by overwriting and updating the low-luminance portion sound velocity mapping data 62 with the high-luminance portion sound velocity mapping data 60. When the in vivo sound velocity of the overwriting mapping data is an invalidated value, the integration processor 32 does not overwrite the value with the invalidated value but maintains the in vivo sound velocity of the mapping data to be overwritten.

When a value of the high-luminance portion sound velocity mapping data 60 and a value of the low-luminance portion sound velocity mapping data 62 are overlapped with each other as the result of the integration process, it is preferable for the integration processor 32 to select the value of the high-luminance portion sound velocity mapping data 60. Generally, the high-luminance tissues are smaller than the low-luminance tissues. Accordingly, if the value of the low-luminance portion sound velocity mapping data 62 is selected for a pixel with the overlapping values, an image of a high-luminance tissue may be hidden by an image of a low-luminance tissue. This may reduce the reception sensitivity and image resolution of the high-luminance tissue. For the low-luminance tissues, even when values of the high-luminance portion sound velocity mapping data 60 are partially applied, the reduction in the reception sensitivity and spatial resolution is limited to the applied portion only, and the reception sensitivity and spatial resolution of the other portions can remain unaffected.

The integration processor 32 may generate a one-dimensional optimum sound velocity sequence (depth-by-depth sound velocity mapping data 72) which indicates an optimum sound velocity in each pixel in the depth direction by averaging the integrated sound velocity mapping data 70 in the scanning direction θ. The integration processor 32 may generate a one-dimensional optimum sound velocity sequence (scan position-by-scan position sound velocity mapping data 74) which indicates an optimum sound velocity in each pixel in the scanning direction θ by averaging the integrated sound velocity mapping data 70 in the depth direction. Further, the integration processor 32 may obtain the overall average 76 of the integrated sound velocity data as a representative value of all the pixels. The integration processor 32 may obtain the depth-by-depth sound velocity mapping data 72, the scan position-by-scan position sound velocity mapping data 74, and the representative value using the median or the maximum of the optimum sound velocities instead of the average. The integration processor 32 may smooth the sound velocities by applying a filter to the sound of velocities of pixels in a case where the difference between the sound velocities of the neighboring pixels is equal to or higher than the threshold in the depth-by-depth sound velocity mapping data 72 or scan position-by-scan position sound velocity mapping data 74.

The integrated sound velocity mapping data 70, the depth-by-depth sound velocity mapping data 72, the scan position-by-scan position sound velocity mapping data 74, and the overall average 76 are supplied to the controller 22. The controller 22 calculates the optimum reception delay data set based on the integrated sound velocity mapping data 70, the depth-by-depth sound velocity mapping data 72, the scan position-by-scan position sound velocity mapping data 74, or the overall average 76. The controller 22 may calculate the reception delay data using a preset sound velocity for a pixel with an invalidated value. In the main scan, the controller 22 supplies the optimum reception delay data set to the receiver 14. The receiver 14 generates reception frames by applying the phasing addition process or the like in accordance with the optimum reception delay data set. The amount of calculation decreases by performing calculation using the averaged depth-by-depth sound velocity mapping data 72, the averaged scan position-by-scan position sound velocity mapping data 74, or the averaged overall average 76, in relation to the calculation of the reception delay data set using the integrated sound velocity mapping data 70 which indicate the in vivo sound velocities of all pixels. Accordingly, the load of the controller 22 decreases. In contrast, when using the integrated sound velocity mapping data 70, the reception delay data set is calculated for each of the pixels. Accordingly, the spatial resolution of an image is improved compared to the calculation using the other sound velocity mapping data.

Further, the sound velocity mapping data for calculating the reception delay data may be selected in accordance with the positional relationship between the tissues included in the scanning surface of the ultrasonic beam. For example, when a high-luminance tissue and a low-luminance tissue are aligned at a certain depth in the scanning direction θ, it is preferable to calculate the reception delay data set based on the scan position-by-scan position sound velocity mapping data 74. This is because, as the scan position-by-scan position sound velocity mapping data 74 indicates the optimum sound velocity at each pixel in the scanning direction θ, it is possible to use the reception delay data set which is suitable for sharpening each of the tissues aligned at a certain depth. The integration processor 32 may perform averaging by changing the direction to average the integrated sound velocity mapping data 70 in accordance with positional relationships of the tissues. The averaging direction may be designated by a user through the operation unit 24.

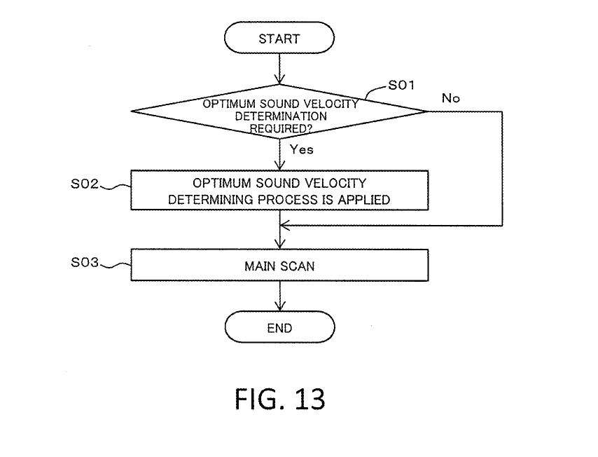

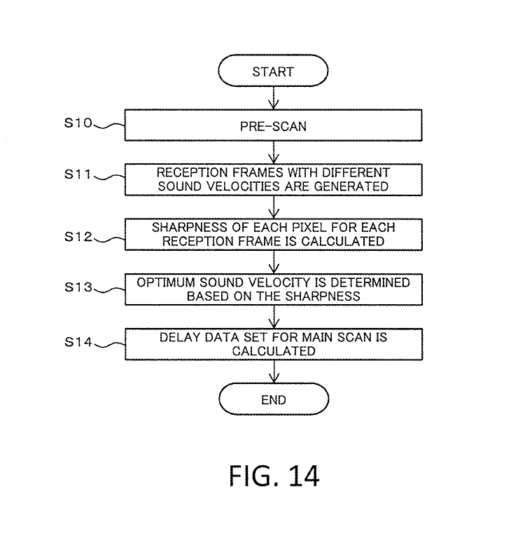

Next, the operations of an ultrasonic diagnostic apparatus according to the present embodiment are described below by referring to FIGS. 13 and 14. FIG. 13 shows the main routine. First, before the main scan (ultrasonic diagnostic), it is determined whether or not to perform an optimum sound velocity determining process (test operation mode) (S01). When a user instructs to perform the optimum sound velocity determining process through the operation unit 24 (Yes in S01), the optimum sound velocity determining process is performed (S02). In Step S02, the processes shown in FIG. 14 described below are performed. The optimum sound velocity is obtained in this way, and thereby a reception delay data set is calculated using the obtained optimum sound velocity. Then, the main scan is performed (S03). In the main scan, the receiver 14 performs the phasing addition process in accordance with the reception delay data set calculated based on the optimum sound velocity. The signal processor 16 and the image forming unit 18 perform processes to form a display frame sequence to display the display frame on the display 20. When it is determined in Step 01 that the optimum sound velocity determining process is not required (No in S01), the main scan is performed. It should be noted that when a user instructs to perform the optimum sound velocity determining process during the main scan, the process in Step S02 may be performed as an interrupt process.

FIG. 14 shows the optimum sound velocity determining process shown in Step S02 in FIG. 13. Before performing the optimum sound velocity determining process, a user positions the probe 10 such that the object will be included on the scanning surface. For example, the user may position the probe 10 while observing the display frames displayed on the display 20. In this example, the high-luminance tissue 52 and the low-luminance tissue 54 shown in FIG. 2 are assumed to be the objects, and thus, the user positions the probe 10 so that these tissues are included on the scanning surface. When the user instructs to perform the optimum sound velocity determination process after the positioning, ultrasonic waves are transmitted and received to perform a pre-scan (S10). For example, two or more reception delay data sets corresponding to the in vivo sound velocities V1 to Vn are supplied from the controller 22 to the receiver 14. The receiver 14 performs processes such as a phasing addition process in accordance with the reception delay data sets. In this way, reception frame sequences corresponding to the in vivo sound velocities V1 to Vn are generated (S11). Then, the optimum sound velocity calculator 26 calculates the sharpness for each of the pixels for each reception frame (S12) and determines the optimum sound velocity of each pixel based on the calculated sharpness (S13). The optimum sound velocity calculator 26 generates the high-luminance portion sound velocity mapping data and the low-luminance portion sound velocity mapping data, both of which indicate the optimum sound velocity, and further generates the integrated sound velocity mapping data, the depth-by-depth sound velocity mapping data, and other data. For an example, the depth-by-depth sound velocity mapping data are supplied to the controller 22, which calculates the reception delay data set for the main scan based on the supplied sound velocity mapping data (S14). Then, the main scan shown in FIG. 13 is performed (step S03).

As described above, in the present embodiment, the sharpness of an image (degree of blur of an image) is calculated based on a luminance waveform in the scanning direction for each frame sequence and determines, as the optimum sound velocity, the in vivo sound velocity corresponding the reception frame with the maximum sharpness. This optimum sound velocity can improve reception delay conditions. As a result, the spatial resolution of an image can be improved. In other words, the sharpness which is calculated based on a luminance waveform reflects the spatial resolution of the image. Accordingly, the identification of the reception frame having the maximum sharpness determines the sound velocity which can improve the spatial resolution of an image.

The calculation and evaluation of the sharpness in consideration of the respective characteristics of high-luminance tissues and low-luminance tissues enable determination of the optimum sound velocity used for sharpening images of high-luminance tissues and low-luminance tissues. A high-luminance tissue appears as a peak portion (convex portion) in the luminance waveform. Therefore, calculation and evaluation of the sharpness by recognizing the peak portion as a single entity allows the determination of the optimum sound velocity for high-luminance tissues. A low-luminance tissue appears as a concave portion in the luminance waveform. Accordingly, respective calculation and evaluation of sharpness of each edge on both sides of the concave portion allows the determination of the optimum sound velocity of low-luminance tissues. This enables generation of the reception delay data sets which are suitable for observing both of the high-luminance tissues and the low-luminance tissues. Therefore, even when two or more tissues of different characteristics are included on the scanning surface, it becomes possible to determine the optimum sound velocity for sharpening images of each tissue and improve the spatial resolution of each tissue.

It should be noted that the high-luminance portion sound velocity calculator 28 may calculate the sharpness by the same calculation method as the low-luminance portion sound velocity calculator 30. Specifically, the high-luminance portion sound velocity calculator 28 may evaluate the sharpness by separately calculating the sharpness of each side of the peak portion.

### Variation 1

Next, Variation 1 is described. In Variation 1, the integration processor 32 selects, as the optimum sound velocity mapping data, the high-luminance portion sound velocity mapping data obtained by the high-luminance portion sound velocity calculator 28 or the low-luminance portion velocity mapping data obtained by the low-luminance portion sound velocity calculator 30.

For example, when only one of the high-luminance tissue and the low-luminance tissue is present on the scanning surface of the ultrasonic beam, the sound velocity mapping data of the other non-existing tissue are not required. In this case, the reception delay data set can be calculated using the sound velocity mapping data corresponding to the existing tissue. For example, when an infiltrating cancer is not present but a calcified tissue is present on the scanning surface, the high-luminance portion sound velocity mapping data should be selected. In contrast, when a calcified tissue is not present but an infiltrating cancer is present on the scanning surface, the low-luminance portion sound velocity mapping data should be selected.

The sound velocity mapping data may be selected by a user or the integration processor 32. In a case where the user selects the sound velocity mapping data, the user may designate the high-luminance tissue or the low-luminance tissue through the operation unit 24. In this way, the sound velocity mapping data corresponding to the designated type of a tissue are selected. The integration processor 32 adopts the sound velocity mapping data selected by the user as the optimum sound velocity mapping data. In a case where the integration processor 32 selects the sound velocity mapping data, the integration processor 32 adopts, as the optimum sound velocity mapping data, the high-luminance mapping data or the low-luminance mapping data which have fewer invalidated pixels. The selected optimum sound velocity mapping data are supplied to the controller 22. The controller 22 calculates the reception delay data based on the optimum sound velocity mapping data.

The integration processor 32 may obtain, based on the selected optimum sound velocity mapping data, the overall average of the depth-by-depth sound velocity mapping data, the scan position-by-scan position sound velocity mapping data, or the optimum sound velocity mapping data. The generated mapping data are supplied to the controller 22, which calculates the reception delay data based on the supplied mapping data.

It should be noted that in the case where the user selects the sound velocity mapping data, the optimum sound velocity calculator 26 may generate the high-luminance portion sound velocity mapping data or the low-luminance portion sound velocity mapping data which have been selected by the user, but not the other unselected sound velocity mapping data.

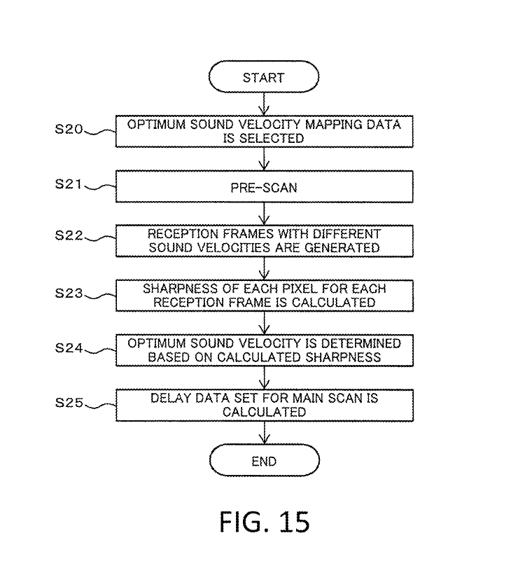

Next, processes according to Variation 1 are described by referring to the flowchart shown in FIG. 15. The processes shown in FIG. 15 correspond to the optimum sound velocity determining process shown in Step S02 in FIG. 13. Prior to the optimum sound velocity determining process, the user selects, through the operation unit 24, the high-luminance portion sound velocity mapping data or the low-luminance portion sound velocity mapping data which are to be used as the optimum sound velocity mapping data (S20). For example, the user may select sound velocity mapping data corresponding to the tissue displayed in the display frame (tissue included on the scanning surface), while observing the display frame displayed on the display 20. Then, similarly as in the above embodiment, a pre-scan is performed (S21); a reception frame sequence corresponding to two or more in vivo sound velocities is generated (S22); the sharpness of each pixel is calculated for each reception frame (S23), and the optimum sound velocity of each pixel is determined in accordance with the sharpness (S24). Then, the optimum sound velocity calculator 26 generates the high-luminance portion sound velocity mapping data and the low-luminance portion sound velocity mapping data. The sound velocity mapping data selected in Step S20 are supplied to the controller 22. The controller 22 calculates the reception delay data set for the main scan based on the selected sound velocity mapping data (S25). Then, the main scan shown in FIG. 13 is performed (Step S03).

In a case where the integration processor 32 selects the optimum sound velocity mapping data, the process of Step S20 is omitted. In this case, the sound velocity mapping data with fewer pixels having an invalidated value are selected by the integration processor 32 and supplied to the controller 22.

As described above, the application of the sound velocity mapping data corresponding to the tissue existing on the scanning surface as the optimum sound velocity mapping data can improve the delay process condition than the application of the integrated sound velocity mapping data in which the high-luminance portion sound velocity mapping data and the low-luminance portion sound velocity mapping data are integrated. In this way, the spatial resolution of an image can be improved.

### Variation 2

Next, Variation 2 is described. In Variation 2, the integration processor 32 counts the pixels with invalidated values in the integrated sound velocity mapping data. When the number of pixels with invalidated values is equal to or greater than a preset threshold, the integration processor 32 outputs invalidation information to the controller 22 indicating that the optimum in vivo sound velocity is invalidated. In this case, the controller 22 supplies to the receiver 14 the reception delay data set which had been used prior to the optimum sound velocity determining process. For example, the controller 22 supplies, to the receiver 14, the reception delay data set based on the default in vivo sound velocity.

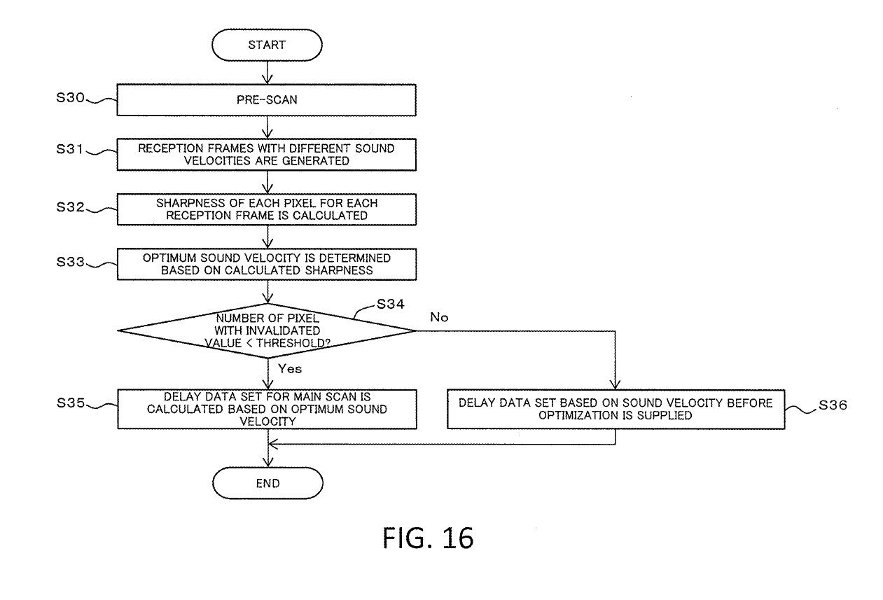

The processes according to Variation 2 are described by referring to the flowchart shown in FIG. 16. The processes shown in FIG. 16 correspond to the optimum sound velocity determining process shown in Step S02 in FIG. 13. As described in the above embodiments, the pre-scan is performed (S30). By the pre-scan, the reception frame sequence corresponding to two or more in vivo sound velocities is generated (S31); the sharpness of each pixel in each reception frame is calculated (S32); and the optimum sound velocity for each pixel is determined based on the calculated sharpness (S33). The integration processor 32 generates the integrated sound velocity mapping data by integrating the high-luminance portion sound velocity mapping data and the low-luminance portion sound velocity mapping data, and counts the number of pixels with invalidated values in the integrated sound velocity mapping data. When the number of pixels with invalidated values is less than the threshold (Yes in S34), the integration processor 32 supplies the integrated sound velocity mapping data to the controller 22. The controller 22 calculates a reception delay data set for the main scan based on the integrated sound velocity mapping data (S35). In contrast, when the number of pixels with invalidated values is equal to or greater than the threshold (No in S34), the integration processor 32 outputs the invalidation information to the controller 22. The controller 22 supplies to the receiver 14 the reception data set which had been used prior to the optimum sound velocity determining process as the reception delay data set for the main scan (S36). Then, the main scan shown in FIG. 13 (Step S03) is performed.

As described above, even when the number of pixels with invalidated values is equal to or greater than the threshold in the integrated sound velocity mapping data, an ultrasonic image of the object can be formed using the reception delay data which had been used prior to the optimum sound velocity determining process. It should be noted that Variations 1, 2 may be combined. In such a case, the integration processor 32 may count the number of pixels with invalidated values in the selected optimum sound velocity mapping data and perform the process (process shown in Step S35 or S36) in accordance with the number of pixels.

Although in the above embodiments and the variations the optimum sound velocity is determined based on the signals after a process applied by the signal processor 16, the optimum sound velocity may be determined based on the signals prior to the process applied by the signal processor 16. Alternatively, the optimum sound velocity may be determined based on the signals after a digital scan conversion.

### REFERENCE SIGNS LIST

10 probe, 12 transmitter, 14 receiver, 16 signal processor, 18 image forming unit, 20 display, 22 controller, 24 operation unit, 26 optimum sound velocity calculator, 28 high-luminance portion sound velocity calculator, 30 low-luminance portion sound velocity calculator, and 32 integration processor.

1. An ultrasonic diagnostic apparatus comprising:
a generator which generates a plurality of frames by repeatedly scanning a subject with an ultrasonic beam;
a pre-scan controller which sequentially sets, to the generator, a plurality of delay process conditions in a trial based on a plurality of tentative sound velocities such that a plurality of tentative frames are generated;
a first waveform analyzer which performs a first waveform analysis for at least one reference data sequence along a preset direction in each of the tentative frames to evaluate a sharpness of an image to obtain a plurality of first waveform analysis results for the plurality of tentative frames;
a second waveform analyzer which performs a second waveform analysis for at least one reference data sequence along a preset direction in each of the tentative frames to evaluate a sharpness of an image to obtain a plurality of second waveform analysis results for the plurality of tentative frames, the second waveform analysis being different from the first waveform analysis;
an optimum sound velocity calculator which calculates an optimum sound velocity based on the plurality of first waveform analysis results and the plurality of second waveform analysis results; and
a main scan controller which sets, to the generator, a delay process condition for a main scan based on the optimum sound velocity.

2. The ultrasonic diagnostic apparatus according to claim 1, wherein
the preset direction is a beam scanning direction;
the first waveform analyzer performs a first local waveform analysis at a plurality of positions in the reference data sequence to obtain a first local waveform analyzed value sequence which forms the first waveform analysis result; and
the second waveform analyzer performs a second local waveform analysis at a plurality of positions in the reference data sequence to obtain a second local waveform analyzed value sequence which forms the second waveform analysis result.

3. The ultrasonic diagnostic apparatus according to claim 2, wherein
the first waveform analyzer performs a first waveform analysis separately for each of the reference data sequences arranged in a depth direction in each of the tentative frames to obtain a first local waveform analyzed value matrix which forms the first waveform analysis result; and
the second waveform analyzer performs a second waveform analysis separately for each of the reference data sequences arranged in a depth direction in each of the tentative frames to obtain a second local waveform analyzed value matrix which forms the second waveform analysis result.

4. (canceled)

5. The ultrasonic diagnostic apparatus according to claim 1, wherein
in the first waveform analysis, a sharpness is analyzed for each convex peak portion; and
in the second waveform analysis, a sharpness is analyzed for each concave low-luminance portion.

6. The ultrasonic diagnostic apparatus according to claim 5, wherein
in the second waveform analysis, gradients of respective edges of the low-luminance portion are separately analyzed and a sharpness of the entire low-luminance portion is analyzed based on the gradients.

7. The ultrasonic diagnostic apparatus according to claim 3, wherein
the optimum sound velocity calculator comprises
a function to generate a first optimum sound velocity map indicating an optimum sound velocity at each position on a beam scanning surface based on the plurality of first local waveform analyzed value matrices corresponding to the plurality of tentative frames; and
a function to generate a second optimum sound velocity map indicating an optimum sound velocity at each position on the beam scanning surface based on the plurality of second local waveform analyzed value matrices corresponding to the plurality of tentative frames,
wherein the optimum sound velocity for the main scan is obtained based on the first optimum sound velocity map and the second optimum sound velocity map.

8. The ultrasonic diagnostic apparatus according to claim 7, wherein
the optimum sound velocity calculator comprises a function to generate a composite map by synthesizing the first optimum sound velocity map and the second optimum sound velocity map.

9. The ultrasonic diagnostic apparatus according to claim 8, wherein
the optimum sound velocity calculator comprises a function to calculate a plurality of optimum sound velocities which define the delay process condition for the main scan by applying an aggregation process to the plurality of optimum sound velocities constituting the composite map.

10. The ultrasound diagnostic apparatus according to claim 1, further comprising:
a first low-pass filter which applies a first filtering process to the plurality of reference data sequences in each of the tentative frames; and
a second low-pass filter which applies a second filtering process to the plurality of reference data sequences in each of the tentative frames, the second filtering process having a stronger effect than the first filtering process,
wherein the first waveform analyzer applies a first waveform analysis to the plurality of reference data sequences in each of the tentative frames after the first filtering process; and
the second waveform analyzer applies a second waveform analysis to the plurality of reference data sequences in each of the tentative frames after the second filtering process.
